(19) United States
(12) Patent Application Publication
(10) Pub. No.: US 2026/0098039 A1
(43) Pub. Date: Apr. 9, 2026

(54) SOLID FORMS OF COMPOUND I OR SALTS THEREOF

(71) Applicant: Jacobio Pharmaceuticals Co., Ltd., Beijing (CN)

(72) Inventors: Haijun LI, Beijing (CN); Mingming CHEN, Beijing (CN); Di KANG, Beijing (CN); Chaojie DANG, Beijing (CN); Amin LI, Beijing (CN); Yanping WANG, Beijing (CN); Wei LONG, Beijing (CN)

(73) Assignee: Jacobio Pharmaceuticals Co., Ltd., Beijing (CN)

(21) Appl. No.: 19/104,027

(22) PCT Filed: Aug. 16, 2023
(86) PCT No.: PCT/CN2023/113244

§ 371 Date: Feb. 14, 2025

Related U.S. Application Data
(63) Continuation of application No. PCT/CN2022/112917, filed on Aug. 17, 2022.

(51) Int. Cl. C07D 471/14; A61K 31/501; A61P 35/00

(52) U.S. Cl. CPC C07D 471/14; A61K 31/501; A61P 35/00

(57) ABSTRACT

The present invention relates to novel forms of Compound I, preparation thereof, pharmaceutical composition containing the same and use thereof, wherein, the compound is (S)-5-((1-(3-(5-methyl-3-(trifluoromethyl)-8,9-dihydropyrido[3′,2′:4,5]pyrrolo[1,2-a]pyrazin-7(6H)-yl)-3-oxopropoxy)propan-2-yl)oxy)-4-(trifluoromethyl)pyridazin-3(2H)-one.

### CROSS-REFERENCES TO RELATED APPLICATION

This application claims the benefit of priority to PCT/CN2022/112917, filed on Aug. 17, 2022 which is hereby incorporated herein by reference in its entirety.

### TECHNICAL FIELD

The present invention relates to solid form of (S)-5-((1-(3-(5-methyl-3-(trifluoromethyl)-8,9-dihydropyrido[3′,2′:4,5]pyrrolo[1,2-a]pyrazin-7(6H)-yl)-3-oxopropoxy)propan-2-yl)oxy)-4-(trifluoromethyl)pyridazin-3(2H)-one or salts thereof, preparation thereof, pharmaceutical composition containing the same and use thereof.

### BACKGROUND OF THE INVENTION

Members of the poly (ADP-ribose) polymerase (PARP) family of enzymes catalyze the post-translational modification of proteins using β-NAD⁺ as a substrate to successively add ADP-ribose moieties onto target proteins: a process termed PARsylation. In the 1960s, this posttranslational modification was first characterized with the identification of PARP1 and its role in DNA repair. Subsequently, additional 16 members of the PARP family were identified, each of which possesses a structurally similar PARP catalytic domain. Furthermore, in addition to its well-studied role in DNA repair, PARsylation has now been shown to modulate processes as diverse as cellular proliferation, apoptosis, DNA methylation, transcriptional regulation and WNT signaling. According to different catalytic activity, PARP family can be divided into three categories: monoPARPS (catalyze the transfer of mono-ADP-ribose units onto their substrates) including the majority of PARP family members; polyPARPS (catalyze the transfer of poly-ADP-ribose units onto their substrates) including PARP1, PARP2, PARP5A, PARP5b; and PARP13 which is the only PARP family member whose catalytic activity could not be demonstrated either in vitro or in vivo.

The monoPARP protein family plays important roles in multiple stress responses associated with the development of cancer, inflammatory diseases, and neurodegenerative diseases. PARP7 as a monoPARP family member has been demonstrated to be overactive in tumors and to play a key role in cancer cell survival. The study found that many cancer cells rely on PARP7 for internal cellular survival, and that PARP7 allows cancer cells to “hide” from the immune system. Inhibition of PARP7 can effectively inhibit the growth of cancer cells and restore interferon signaling, effectively prevent cancer cells from evading the immune system, and inhibiting the “brake” of innate and adaptive immune mechanisms. In several cancer models, PARP7 inhibitors exhibit persistent tumor growth inhibition, potent anti-proliferative activity, and interferon signaling restoration.

Moreover, the solid form of a compound is vital to a medicine as is known in the art. Thus improved properties are required for the development of the compound, such as higher physical and chemical stability against high temperature, high humidity and/or light exposure to maintain the quality of medicine containing the Compound I as active ingredient, and/or higher absorption to achieve good therapeutic use when the Compound I is administered orally. Therefore, it is desired to develop new crystalline form of Compound I to meet these needs.

### BRIEF SUMMARY OF THE INVENTION

The compound named (S)-5-((1-(3-(5-methyl-3-(trifluoromethyl)-8,9-dihydropyrido[3′,2′:4,5]pyrrolo[1,2-a]pyrazin-7(6H)-yl)-3-oxopropoxy)propan-2-yl)oxy)-4-(trifluoromethyl)pyridazin-3(2H)-one having the following structure, referred herein as Compound I, is a potent PARP7 inhibitor with excellent activity against cancer related to PARP7.

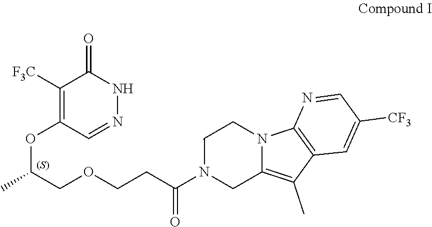

In one aspect, provided herein is a form of Compound I, which is selected from: a crystalline of Compound I; a salt of Compound I with an acid; and a crystalline of the salt of Compound I with an acid.

In one aspect, provided herein is a process to prepare the crystalline Form 1 of Compound I.

In one aspect, provided herein is a process to prepare the crystalline Form A of the salt of Compound I with an acid.

In one aspect, provided herein is a pharmaceutical composition comprising the form of Compound I.

In another aspect, provided herein is use of the form of Compound I for the manufacture of a medicament for the treatment of a cancer related to PARP7.

In another aspect, provided herein is a method of treating a subject having cancer related to PARP7, comprising administering an effective amount of the form of Compound I to the subject.

### BRIEF DESCRIPTION OF THE DRAWINGS

FIG. 1: The XRPD pattern of Compound I of the crystalline Form 1 prepared in example 2.

FIG. 2: The TGA plot of Compound I of the crystalline Form 1 prepared in example 2.

FIG. 3: The DSC thermogram of Compound I of the crystalline Form 1 prepared in example 2.

FIG. 4: The DVS plot of Compound I of the crystalline Form 1 prepared in example 2.

FIG. 5: The sorption isothermal curve plot of Compound I of the crystalline Form 1 prepared in example 2.

FIG. 6: The compared XRPD Plots of Solids in Equilibrium Solubility Test on Compound I of the crystalline Form 1 prepared in example 2.

FIG. 7: Another compared XRPD Plots of Solids in Equilibrium Solubility Test on Compound I of the crystalline Form 1 prepared in example 2.

FIG. 8: The compared DSC Plot of Stability Study on Compound I of the crystalline Form 1 prepared in example 2.

FIG. 9: Another compared DSC Plot of Stability Study on Compound I of the crystalline Form 1 prepared in example 2.

FIG. 10: The XRPD pattern of the amorphous Compound I prepared in example 5.

FIG. 11: The XRPD pattern of the crystalline Form A of tosilate of Compound I prepared in example 6.

FIG. 12: The TGA plot of the crystalline Form A of tosilate of Compound I prepared in example 6.

FIG. 13: The DSC thermogram of the crystalline Form A of tosilate of Compound I prepared in example 6.

FIG. 14: The PLM plot of the crystalline Form A of tosilate of Compound I prepared in example 6.

FIG. 15: The ¹H-NMR plot of the crystalline Form A of tosilate of Compound I prepared in example 6.

FIG. 16: The Hygroscopicity XRPD pattern of the tosilate Form A of tosilate of Compound I prepared in example 6.

### DETAILED DESCRIPTION OF THE INVENTION

In the following description, certain specific details are set forth in order to provide a thorough understanding of various embodiments of the invention. However, one skilled in the art will understand that the invention may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### Definitions

The compound name of the present invention is named according to IUPAC rules or using ChemBioDraw Ultra and one skilled in the art understands that the compound structure may be named or identified using other commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC). Accordingly, the Compound I having the above structure may also be named or identified as (S)-5-((1-(3-(5-methyl-3-(trifluoromethyl)-8,9-dihydropyrido[3′,2′:4,5]pyrrolo[1,2-a]pyrazin-7(6H)-yl)-3-oxopropoxy)propan-2-yl)oxy)-4-(trifluoromethyl)pyridazin-3(2H)-one.

Unless indicated otherwise, the word “comprise” and variations thereof, such as, “comprises” and “comprising” used herein are to be construed as “including, but not limited to” in an opened, inclusive sense. In other words, the other element(s) not specifically disclosed or listed may be included. The terms “comprising” includes “consisting essentially (substantially) of”. The term “consisting essentially (substantially) of” includes “consisting of”.

Unless indicated otherwise, the singular form “a”, “an” and “the” used herein include plural references.

Unless indicated otherwise, the term “about” used herein means having a value falling within an accepted standard of error of the specific value, when considered by one of ordinary skill in the art.

Reference throughout this specification to “one embodiment” or “an embodiment” or “embodiments” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless indicated otherwise, the term “room temperature” used herein means the temperature range of the external circumstances is 10-30° C.

Unless indicated otherwise, the term “substantially” when referring, for example, to ¹H-NMR spectrum, an XRPD pattern, a DSC thermogram or TGA plot, includes a pattern, thermogram or plot that is not necessarily identical to those depicted herein, but that falls within the limits of experimental error or deviations when considered by one of ordinary skill in the art. For instance, the term “substantially the same” means that variability typical for a particular method is taken into account. For example, with reference to X-ray diffraction peak positions, the term “substantially the same” means that typical variability in peak position and intensity are taken into account. One skilled in the art will appreciate that the peak positions (2θ) will show some variability, typically as much as ±0.2°. Further, one skilled in the art will appreciate that relative peak intensities will show inter-apparatus variability as well as variability due to degree of crystallinity, preferred orientation, prepared sample surface and other factors known to those skilled in the art and should be taken as qualitative measures only.

Unless indicated otherwise, the term “X-ray powder diffraction (XRPD) pattern” as used herein refers to a diffraction pattern observed by an experiment or a parameter derived therefrom. The X-ray powder diffraction pattern is characterized by the peak position and/or the peak intensity. The characteristic peaks of a given XRPD can be selected according to the peak locations and their relative intensity to conveniently distinguish this crystalline structure from others. The XRPD pattern in the present invention is obtained using the Bruker D8 Advance Diffractometer. Those skilled in the art recognize that the measurements of the XRPD peak locations and/or intensity for a given crystalline form of the same compound will vary within a margin of error. The values of degree 2θ allow appropriate error margins in the present invention. Typically, the error margins are represented by “±”. For example, the degree 2θ of about “8.88±0.2°” denotes a range from about 8.88+0.2, i.e., about 9.08, to about 8.88−0.2, i.e., about 8.68.

Unless indicated otherwise, the term “Differential Scanning Calorimeter (DSC) thermogram” as used herein refers to a thermogram by the differential scanning calorimeter.

Unless indicated otherwise, the term “Thermogravimetric Analysis (TGA) thermogram” as used herein refers to a plot by the thermogravimetric analytical instrument.

Unless indicated otherwise, the term “Dynamic Vapor Sorption (DVS) plot” or “Sorption Isothermal plot” as used herein refers to a plot by the dynamic vapor sorption instrument.

Unless indicated otherwise, the term “Polarized light microscope (PLM) gram” as used herein refers to a graph by the polarized light microscope instrument.

Unless indicated otherwise, the term “Fourier Transform Infra-Red (FT-IR) gram” as used herein refers to a graph by the Fourier Transform Infra-Red Spectromete.

Unless indicated otherwise, the term “anhydrous” as used herein, refers to a crystalline form containing less than about 1% (w/w) of adsorbed moisture as determined by standard methods, such as a Karl Fisher analysis.

Unless indicated otherwise, the term “effective amount” as used herein, refers to that amount of a therapeutic compound necessary or sufficient to perform its intended function within a mammal. An effective amount of the therapeutic compound can vary according to factors such as the amount of the causative agent already present in the mammal, the age, sex, and weight of the mammal.

Unless indicated otherwise, the term “treat”, “treating” or “treatment” as used herein in connection to a disease or disorder refers in some embodiments, to alleviating or ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g, stabilization of a physical parameter), or both. In yet another embodiment, “treat”, “treating” or “treatment” refers to preventing or delaying the onset or development or progression of the disease or disorder or symptom thereof.

Unless indicated otherwise, the term “subject” or “patient” as used herein, refers to human and non-human mammals, including but, not limited to, primates, rabbits, pigs, horses, dogs, cats, sheep, and cows. In particular embodiments, a subject or patient is a human. In some embodiments, the term “patient” or “subject” refers to a human being who is diseased with the condition (i.e., disease or disorder) described herein and who would benefit from the treatment. As used herein, a subject is “in need of” a treatment if such subject (patient) would benefit biologically, medically or in quality of life from such treatment.

Unless indicated otherwise, the term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Unless indicated otherwise, all ingredient concentrations are presented in units of % weight/volume (% w/v). As is commonly understood, the % w/v value refers to the amount of the particular component or ingredient in the formulation. It is commonly understood that equivalent concentrations can be expressed in different units. For example, a concentration of 0.1% w/v can also be expressed as a 1 mg/ml solution.

Unless otherwise specified, the weight or dosage referred to herein for the crystalline forms of salts of Compound I is the weight or dosage of the Compound I itself, not that of a salt thereof. The weight or dosage of a corresponding salt of the Compound I suitable for the methods or compositions disclosed herein may be calculated based on the ratio of the molecular weights of the salt and the Compound I itself.

In one aspect, provided herein is a form of Compound I, which is selected from:
A crystalline of Compound I;
A salt of Compound I with an acid; and
A crystalline of the salt of Compound I with an acid;
Wherein, the Compound I is:

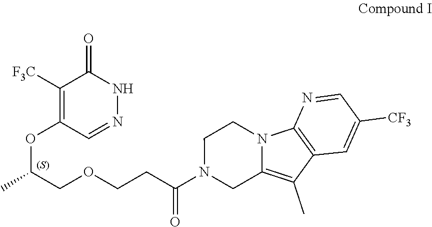

The acid is selected from hydrochloric acid, sulfuric acid, hydrobromic acid, methanesulfonic acid, tosic acid (p-toluenesulfonic acid), oxalic acid, maleic acid, phosphoric acid, L-tartric acid, fumaric acid, citric acid, lactobionic acid, mandelic acid, L-malic acid, hippuric acid, L-lactic acid, succinic acid, benzoic acid, adipic acid, acetic acid.

In some embodiments, the crystalline of Compound I is crystalline Form 1 of Compound I, and the crystalline Form 1 of Compound I is characterized by X-ray powder diffraction pattern comprising characteristic peaks at 2θ values of 8.12±0.2°, 11.96±0.2°, 13.48±0.2° and 15.27±0.2°.

In some embodiments, the crystalline Form 1 of Compound I is characterized by X-ray powder diffraction pattern further comprising one or more characteristic peaks at 2θ values selected from 16.11±0.2°, 16.49±0.2°, 19.79±0.2° and 20.30±0.2°.

In some embodiments, the crystalline Form 1 of Compound I is characterized by X-ray powder diffraction pattern further comprising:
one or two characteristic peaks at 2θ value selected from 19.79±0.2° and 20.30±0.2°, and
one or two characteristic peaks at 2θ values selected from 16.11±0.2° and 16.49±0.2°.

In some embodiments, the crystalline Form 1 of Compound I is characterized by X-ray powder diffraction pattern further comprising characteristic peaks at 2θ value selected from:
16.11±0.2° and 16.49±0.2°;
16.11±0.2°, 16.49±0.2° and 20.30±0.2°;
16.11±0.2°, 16.49±0.2° and 19.79±0.2°;
19.79±0.2° and 20.30±0.2°;
16.11±0.2°, 19.79±0.2° and 20.30±0.2°; or
16.49±0.2°, 19.79±0.2° and 20.30±0.2°.

In some embodiments, the crystalline Form 1 of Compound I is characterized by X-ray powder diffraction pattern comprising characteristic peaks at 2θ values of 8.12±0.2°, 11.96±0.2°, 13.48±0.2°, 15.27±0.2°, 16.11±0.2°, 16.49±0.2°, 19.79±0.2° and 20.30±0.2°.

In some embodiments, the crystalline Form 1 of Compound I is characterized by X-ray powder diffraction pattern further comprising one or two characteristic peaks at 2θ values selected from 12.49±0.2° and 21.45±0.2°.

In some embodiments, the crystalline Form 1 of Compound I is characterized by X-ray powder diffraction pattern comprising characteristic peaks at 2θ values of 8.12±0.2°, 11.96±0.2°, 12.49±0.2°, 13.48±0.2°, 15.27±0.2°, 16.11±0.2°, 16.49±0.2°, 19.79±0.2°, 20.30±0.2° and 21.45±0.2°.

In some embodiments, the crystalline Form 1 of Compound I is characterized by X-ray powder diffraction pattern further comprising one or two characteristic peaks at 2θ values selected from 22.59±0.2° and 24.06±0.2°.

In some embodiments, the crystalline Form 1 of Compound I is characterized by X-ray powder diffraction pattern comprising the characteristic peaks at 2θ values of 8.12±0.2°, 11.96±0.2°, 12.49±0.2°, 13.48±0.2°, 15.27±0.2°, 16.11±0.2°, 16.49±0.2°, 19.79±0.2°, 20.30±0.2°, 21.45±0.2°, 22.59±0.2° and 24.06±0.2°.

In some embodiments, the crystalline Form 1 of Compound I is characterized by X-ray powder diffraction pattern comprising characteristic peaks at 2θ values of the following Table 1:

TABLE 1

| 2 theta | d spacing | Intensity % |
|---|---|---|
| 8.12 | 10.88 | 5.4 |
| 11.96 | 7.40 | 100 |
| 12.49 | 7.08 | 13.8 |
| 13.48 | 6.56 | 30.7 |
| 15.27 | 5.80 | 58.7 |
| 16.11 | 5.50 | 12.5 |
| 16.49 | 5.37 | 12.1 |
| 19.79 | 4.48 | 25.4 |
| 20.30 | 4.37 | 35 |
| 21.45 | 4.14 | 65.2 |
| 22.59 | 3.93 | 83 |
| 24.06 | 3.70 | 58.4 |

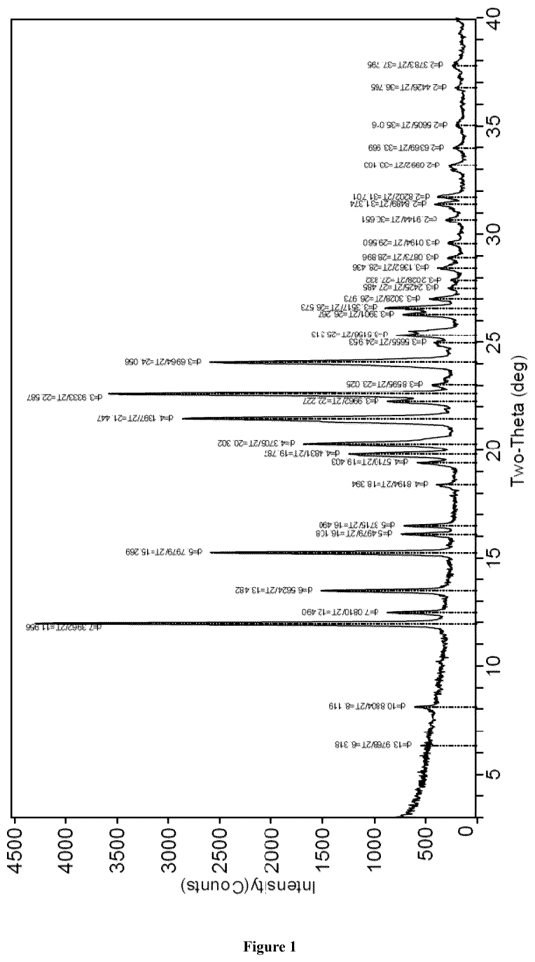

In some embodiments, the crystalline Form 1 of Compound I is characterized by the X-ray powder diffraction pattern which is the same as FIG. 1.

In some embodiments, the acid is tosic acid (p-toluenesulfonic acid).

In some embodiments, the crystalline Form A of the salt of Compound I with an acid is characterized by X-ray powder diffraction pattern comprising characteristic peaks at 2θ values of 6.74±0.2°, 9.12±0.2°, 14.75±0.2° and 15.93±0.2°.

In some embodiments, the crystalline Form A of the salt of Compound I with an acid is characterized by X-ray powder diffraction pattern further comprising one or more characteristic peaks at 2θ values selected from 11.94±0.2°, 12.73±0.2°, 15.26±0.2° and 16.55±0.2°.

In some embodiments, the crystalline Form A of the salt of Compound I with an acid is characterized by X-ray powder diffraction pattern further comprising:
one or two characteristic peaks at 2θ value selected from 11.94±0.2° and 12.73±0.2°, and
one or two characteristic peaks at 2θ values selected from 15.26±0.2° and 16.55±0.2°.

In some embodiments, the crystalline Form A of the salt of Compound I with an acid is characterized by X-ray powder diffraction pattern comprising characteristic peaks at 2θ values of 6.74±0.2°, 9.12±0.2°, 11.94±0.2°, 12.73±0.2°, 14.75±0.2°, 15.26±0.2°, 15.93±0.2° and 16.55±0.2°.

In some embodiments, the crystalline Form A of the salt of Compound I with an acid is characterized by X-ray powder diffraction pattern further comprising one or two characteristic peaks at 2θ values selected from 18.24±0.2°, 21.09±0.2° and 22.25±0.2°.

In some embodiments, the crystalline Form A of the salt of Compound I with an acid is characterized by X-ray powder diffraction pattern comprising characteristic peaks at 2θ values of 6.74±0.2°, 9.12±0.2°, 11.94±0.2°, 12.73±0.2°, 14.75±0.2°, 15.26±0.2°, 15.93±0.2°, 16.55±0.2°, 18.24±0.2°, 21.09±0.2° and 22.25±0.2°.

In some embodiments, the crystalline Form A of the salt of Compound I with an acid is characterized by X-ray powder diffraction pattern which is the same as FIG. 11.

In one aspect, provided herein is a process of preparing the crystalline Form 1 of Compound I, comprising dissolving Compound I in ethanol; adding water to precipitate solids; and filtered.

In one aspect, provided herein is a process of preparing the crystalline Form A of the salt of Compound I with an acid, comprising dissolving Compound I in ethyl acetate to obtain solution 1; dissolving tosic acid (p-toluenesulfonic acid) in ethanol to obtain solution 2; adding solution 2 to solution 1, to obtain solution 3; adding n-Heptane and stirring until precipitation occurred, then continuing stirring and precipitation do not disappeared; centrifuging the precipitation and dried to obtain the crystalline Form A of the salt of Compound I with an acid.

In one aspect, provided herein is a pharmaceutical composition comprising a therapeutically effective amount of the form of Compound I, and at least one pharmaceutically acceptable excipient.

In one aspect, provided herein is use of the form of Compound I or the pharmaceutical composition for the manufacture of a medicament for the treatment of a cancer related to PARP7.

In one aspect, provided herein is a method of treating a subject having a cancer related to PARP7, said method comprising administering to the subject a therapeutically effective amount of the form of Compound I or the pharmaceutical composition.

In one aspect, provided herein is the form of Compound I or the pharmaceutical composition for use in the treatment of a cancer related to PARP7.

In some embodiments, the cancer related to PARP7 is PARP7 overexpression associated cancer.

In some embodiments, the cancer is selected from breast cancer, cancer of the central nervous system, endometrium cancer, kidney cancer, large intestine cancer, lung cancer, esophagus cancer, tongue cancer, ovarian cancer, pancreatic cancer, prostate cancer, stomach cancer, mesothelioma, melanoma, fibrosarcoma, bladder cancer, rectal cancer, lymphoma, cervical cancer, head and neck cancer, upper aerodigestive cancer, colorectal cancer, urinary tract cancer, or colon cancer; More preferably, each cancer is independently selected from adenocarcinoma, squamous cell carcinoma, mixed adenosquamous carcinoma, undifferentiated carcinoma; More preferably, the ovarian cancer comprises high grade ovarian serious adenocarcinoma, ovarian mucinous cystadenocarcinoma or malignant ovarian Brenner tumor; the kidney cancer comprises clear cell renal cell carcinoma; the tongue cancer comprises tongue squamous cell carcinoma; the lung cancer comprises lung adenocarcinoma, lung adenosquamous carcinoma, squamous cell lung carcinoma, large cell lung carcinoma, small cell lung carcinoma, papillary adenocarcinoma of the lung or non-small cell lung carcinoma; the pancreatic cancer comprises pancreatic adenocarcinoma or pancreatic ductal adenocarcinoma; the esophagus cancer comprises esophageal squamous cell carcinoma; the mesothelioma comprises biphasic mesothelioma; the cancer of the central nervous system comprises neuroglioma, glioblastoma or glioblastoma multiforme; the stomach cancer comprises gastric adenocarcinoma; the breast cancer comprises ductal breast carcinoma, breast adenocarcinoma or HR+ breast cancer; the bladder cancer comprises bladder squamous cell carcinoma; the melanoma comprises malignant melanoma; the colon cancer comprises colon adenocarcinoma; the head and neck cancer comprises head and neck small squamous cell cancer.

### EXAMPLES

The following Examples are provided to better illustrate the present invention. All parts and percentages are by weight and all temperatures are degrees Celsius, unless explicitly stated otherwise. The following abbreviations in the Table 2 have been used in the examples:

TABLE 2

| Abbreviation | Meaning |
|---|---|
| EtOH | Ethanol |
| XRPD | X-ray powder diffraction |
| DSC | Differential scanning calorimetry |
| TGA | Thermal gravimetric analysis |
| DVS | Dynamic vapor sorption |
| PLM | Polarized light microscope |
| FT-IR | Fourier Transform Infra-Red |
| | Spectromete |
| IV | Intravenous administration |
| P.O./Per os | Oral administration |
| Cl_obs | Observed clearance from plasma |
| F | Bioavailability |
| Vss_obs | Observed volume of distribution at |
| | steady state |
| AUCₗₐₛₜ | Area under the plasma |
| | concentration-time curve from time zero to time |
| | of last measurable concentration |
| AUC_{inf} | Area under the plasma concentration-time |
| | curve from time zero to |
| | infinity |

### Example 1. Synthesis and Pharmacological Testing of Compound I

### Synthesis of (S)-5-((1-(3-(5-methyl-3-(trifluoromethyl)-8,9-dihydropyrido[3′,2′:4,5]pyrrolo[1,2-a]pyrazin-7(6H)-yl)-3-oxopropoxy)propan-2-yl)oxy)-4-(trifluoromethyl)pyridazin-3(2H)-one (Compound I)

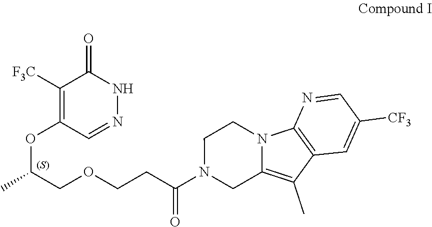

### Step 1: Synthesis of Intermediate A1 (INT A1)

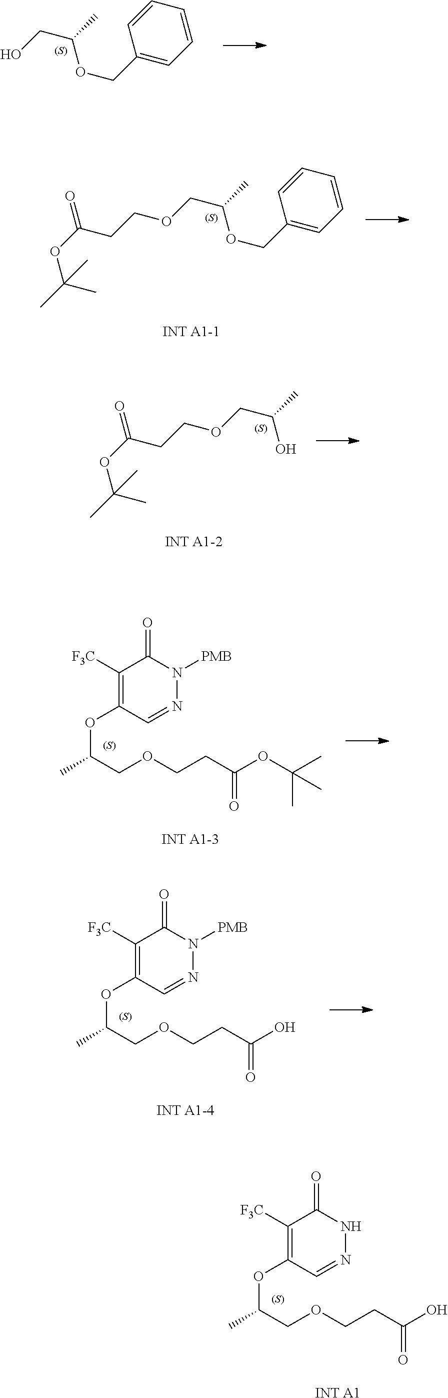

(S)-2-(benzyloxy)propan-1-ol (21.33 g, 128.33 mmol, 1.0 eq.), tert-butyl acrylate (70.84 g, 552.71 mmol, 4.31 eq.) and Cs₂CO₃ (125.61 g, 385.52 mmol, 3.00 eq.) were dispersed in DMSO (210 mL). The reaction mixture was stirred for 3 hrs at room temperature, poured into water (200 mL) and extracted with EA (200 mL×3). The organic layers were combined, dried over anhydrous Na₂SO₄, and then concentrated under reduced pressure to obtain a residue which was purified with Prep-HPLC (C18 column, eluted with H₂O/CH₃CN) to afford INT A1-1 (26.52 g). LCMS: m/z=295 [M+1]⁺.

A mixture of INT A1-1 (10.71 g, 36.38 mmol, 1.0 eq.), Pd/C (1.02 g, 9.58 mmol, 0.26 eq.) and MeOH (10 mL) was purged and maintained with an inert atmosphere of hydrogen, stirred for 48 hrs at room temperature, and then filtered. The filtrate was concentrated under reduced pressure to afford a crude product (9.15 g) containing INT A1-2 which was used in next step without further purification. LCMS: m/z=205 [M+1]⁺.

In an atmosphere of nitrogen, INT A1-2 (9.15 g, 44.80 mmol, 1.09 eq.), 5-chloro-2-(4-methoxybenzyl)-4-(trifluoromethyl)pyridazin-3(2H)-one (13.11 g, 41.14 mmol, 1.0 eq.) and t-BuONa (5.52 g, 57.44 mmol, 1.40 eq.) were dispersed in DCM (50 mL). The reaction mixture was stirred for 2 hrs at room temperature, washed with NH₄Cl(aq.) and then extracted with DCM (50 mL×3). The organic layers were combined, dried over anhydrous Na₂SO₄, and then concentrated under reduced pressure to obtain a residue which was purified with Prep-HPLC (C18 column, eluted with H₂O/CH₃CN) to afford INT A1-3 (12.81 g). LCMS: m/z=487 [M+1]⁺.

TFA (10 mL) was added dropwise at room temperature to a solution of INT A1-3 (12.81 g, 26.33 mmol, 1.0 eq.) dissolved in DCM (40 mL). The reaction mixture was stirred for 2 hrs at room temperature, quenched with saturated NaHCO₃ aqueous solution (50 mL), and extracted with of EA (100 mL×3). The organic layers were combined, dried over anhydrous Na₂SO₄, and then concentrated under reduced pressure to afford a crude product (11.33 g) of INT A1-4 which was used in next step without further purification. LCMS: m/z=431 [M+1]⁺.

TfOH (30 mL) was added dropwise at room temperature to a solution of INT A1-4 (12.81 g, crude) dissolved in TFA (200 mL). The reaction mixture was stirred for 2 hrs at room temperature, quenched with saturated NaHCO₃ aqueous solution (850 mL), and then extracted with of EA (500 mL×3). The organic layers were combined, dried over anhydrous Na₂SO₄, and then concentrated under reduced pressure to obtain a residue which was purified with Prep-HPLC (C18 column, eluted with H₂O/CH₃CN) to afford INT A1 (5.23 g, yield 64%). LCMS: m/z=311 [M+1]f.

### Step 2: Synthesis of Intermediate B1 (INT B1)

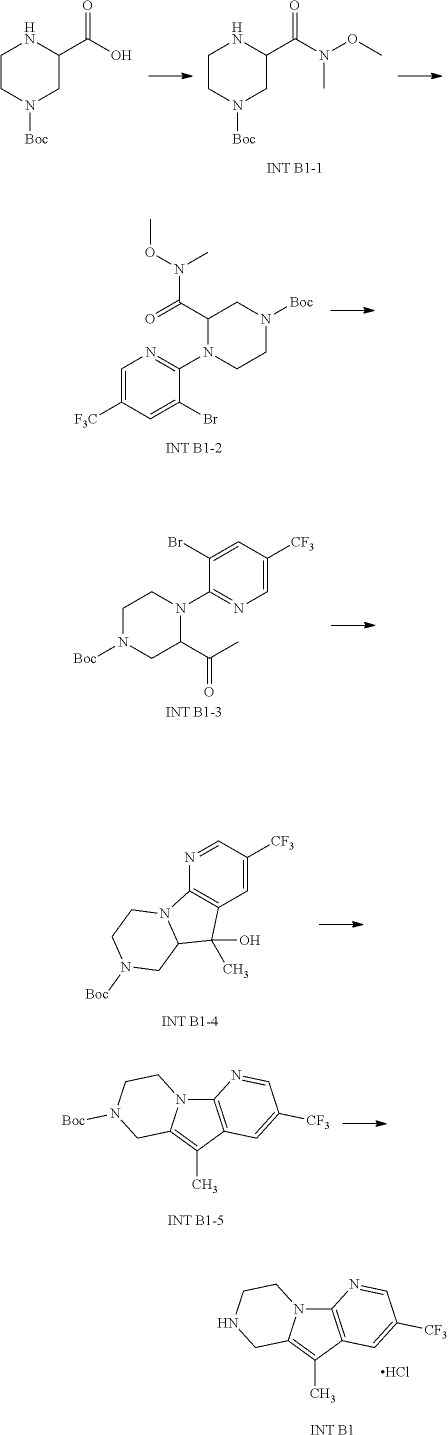

4-(tert-butoxycarbonyl)piperazine-2-carboxylic acid (21.59 g, 93.76 mmol, 1.0 eq.), N,O-dimethylhydroxylamine hydrochloride (21.55 g, 220.93 mmol, 2.36 eq.), DIPEA (42.43 g, 328.30 mmol, 3.50 eq.) and HATU (43.87 g, 115.38 mmol, 1.23 eq.) were dispersed in CH₃CN (200 mL). The reaction mixture was stirred for 3 hrs at room temperature and then concentrated under reduced pressure to obtain a residue which was purified with Prep-HPLC (C18 column, eluted with H₂O/CH₃CN) to afford INT B1-1 (12.68 g, yield 49%). LCMS: m/z=274 [M+1]⁺.

3-bromo-2-fluoro-5-(trifluoromethyl)pyridine (19.09 g, 78.24 mmol, 1.25 eq.), INT B1-1 (17.10 g, 62.56 mmol, 1.0 eq.) and DIPEA (9.22 g, 71.34 mmol, 1.14 eq.) were dispersed in DMF (100 mL) at room temperature. The reaction mixture was stirred for 16 hrs at 80° C., poured into water (100 mL), and extracted with DCM (100 mL×3). The organic layers were combined and concentrated under reduced pressure to obtain a residue which was purified with silica gel column (eluted with Hex/EA) to afford INT B1-2 (15.59 g, yield 50%). LCMS: m/z=497, 499 [M+1]⁺.

In an atmosphere of nitrogen, MeMgBr (14 mL, 42 mmol, 1.54 eq.) was added at −20° C. to a solution of INT B1-2 (13.59 g, 27.33 mmol, 1.0 eq.) dissolved in THF (140 mL). The reaction mixture was stirred for 3 hrs at −20° C., quenched with saturated NH₄Cl aqueous solution (200 mL), and extracted with EA (200 mL×3). The organic layers were combined and then concentrated under reduced pressure to obtain a residue which was purified with Prep-HPLC (C18 column, eluted with H₂O/CH₃CN) to afford INT B1-3 (10.9 g, yield 88%). LCMS: m/z=452, 454 [M+1]⁺.

In an atmosphere of nitrogen, n-BuLi (14 mL, 42.0 mmol, 1.74 eq.) was added dropwise at −78° C. to a solution of INT B1-3 (10.9 g, 24.10 mmol, 1.0 eq.) dissolved in THF (100 mL). The reaction mixture was stirred for 1 h at −78° C., quenched with saturated NH₄Cl aqueous solution (200 mL), and then extracted with EA (200 mL×3). The organic layers were combined and then concentrated under reduced pressure to obtain a residue which was purified with Prep-HPLC (C18 column, eluted with H₂O/CH₃CN) to afford INT B1-4 (2.76 g, yield 30%). LCMS: m/z=374 [M+1]⁺.

A mixture of INT B1-4 (6.19 g, 16.58 mmol, 1.0 eq.), Et₃N (3.69 g, 36.47 mmol, 2.20 eq.), DMAP (122 mg, 0.99 mmol, 0.06 eq.) and DCM (100 mL) was cooled to 0° C., and then MsCl (2.94 g, 25.67 mmol, 1.55 eq.) was added dropwise. The reaction mixture was stirred for 1 h at 0° C., poured into saturated NaHCO₃ aqueous solution (100 mL), and then extracted with DCM (100 mL×3). The organic layers were combined and then concentrated under reduced pressure to obtain a residue which was purified with silica gel column (eluted with Hex/EA) to afford INT B1-5 (5.40 g, yield 91%). LCMS: m/z=356 [M+1]⁺.

A mixture of INT B1-5 (5.05 g, 14.21 mmol, 1.0 eq.) and HCl/1,4-dioxane (100 mL, 1 N) was stirred for 3 hrs at room temperature and then concentrated under reduced pressure to afford a crude product (6.72 g) of a hydrochloride of INT B1 which was used in next step without further purification. LCMS: m/z=256 [M+1]⁺.

### Step 3: Synthesis of Compound I

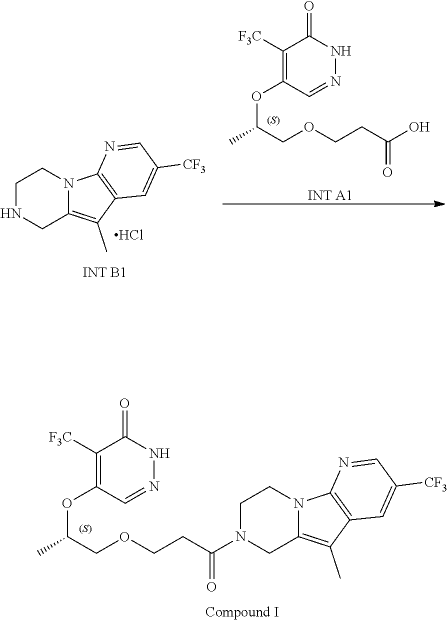

INT A1 (99 mg, 0.32 mmol, 1.0 eq.), INT B1 (104 mg, 0.36 mmol, 1.13 eq.), and TEA (194 mg, 1.92 mmol, 6.00 eq.) were dissolved in THF (1 mL) to form a solution, and then T₃P (312 mg, 0.98 mmol, 3.06 eq.)(50% in EA) was added to the solution. The reaction mixture was stirred for 1 h at room temperature. The resulting solution was diluted with water (2 mL), extracted with EA (2×3 mL) and the organic layer was combined, washed with brine (3 mL) and concentrated under vacuum. The residue was purified with Prep-HPLC (C18 column, eluted with H₂O/CH₃CN) and evaporate under reduced pressure to afford Compound I (149.5 mg, 85% yield) as white foam. LCMS: m/z=548 [M+1]⁺.

¹H NMR (400 MHz, CD₃OD) δ 8.43 (s, 1H), 8.18 (s, 1H), 8.14 (d, J=19.8 Hz, 1H), 5.12-4.98 (m, 1H), 4.89-4.96 (m, 2H), 4.23-4.30 (m, 2H), 4.11-3.98 (m, 2H), 3.90-3.81 (m, 1H), 3.77 (m, 1H), 3.72-3.54 (m, 2H), 2.76 (t, 2H), 2.27 (d, J=6.2 Hz, 3H), 1.27-1.32 (m, 3H).

### Pharmacological Testing

### 1. PARP7 Enzymatic Experiments

The PARP7 enzyme inhibitory activity of each compound was tested using HTRF (homogeneous time resolved fluorescence) assay, and the half inhibitory concentration IC₅₀ thereof was obtained.

(1) Each compound to be tested was prepared using gradient dilution method with DMSO and water to obtain a solution with the concentration of 50 nM, 10 nM, 2 nM, 0.4 nM, and 0.08 nM. The concentration of DMSO in the solution of each compound to be tested was 2%.

(2) PARP7 enzyme (Cell Chemical Biology 27, 877-887, Jul. 16, 2020; the fusion tags was N-His6-TEV-AviMHHHHHHSSGVDLGTENLYFQSNAGLNDIFEAQKIEWHE) was dissolved in the buffer solution (the pH of the buffer solution was 7.4, and the buffer solution contained 25 mM HEPES (N-(2-hydroxyethyl) piperazine-N′-2-sulfonic acid), 120 mM NaCl, 5 mM MgCl₂, 2 mM DTT (Dithiothreitol), 0.002% (ml/ml) Tween-20, 0.1% (ml/ml) BSA (bovine serum albumin) and water) to obtain a PARP7 enzyme solution with the concentration of 6 nM.

(3) The RBN011147 (Cell Chemical Biology 27, 877-887, Jul. 16, 2020), MAb Anti His-Tb cryptate Gold (Cisbio, Cat. No 61GSTTLF, Lot. No 09A), and Streptavidin-d2 (Cisbio, Cat. No 610SADLF, Lot. No 19G) were diluted with buffer solution (the pH of the buffer solution was 7.4, and the buffer solution contained 25 mM HEPES (N-(2-hydroxyethyl) piperazine-N′-2-sulfonic acid), 120 mM NaCl, 5 mM MgCl₂, 2 mM DTT (Dithiothreitol), 0.002% (ml/ml) Tween-20, 0.1% (ml/ml) BSA (bovine serum albumin) and water) to obtain the solution containing fluorophore with the concentration of 10 nM, 0.7 nM, and 2.5 nM respectively. The MAb Anti His-Tb cryptate Gold was the donor fluorophore, and the Streptavidin-d2 was the acceptor fluorophore.

(4) 2.5 μl of the solution of the compound to be tested was transferred into 384-well plate, 2.5 μl of the PARP7 enzyme solution was added. The resulting solution was incubated for 15 mins, and then 5 μl of the solution containing fluorophore was added. The resulting mixture was incubated at 25° C. for 3 hrs to obtain the final solution to be tested.

(5) The fluorescence signal was read on SPARK plate reader (Tecan), the wavelength of the excitation spectrum of the SPARK plate reader was 320 nm, and the wavelength of the emission spectrum of the SPARK plate reader was 620 nm and 665 nm. The ratio of absorbance at 620 nm to absorbance at 665 nm was calculated for the solution in each well. The ratio was calculated according to the following formula: Ratio=absorbance at 665 nm/absorbance at 620 nm×10⁴.

(6) The activation of the compounds to be tested was calculated according to the following formula: Activation (%)=100× (ratio_{compound}−ration_{negative})/(ratioₚₒₛᵢₜᵢᵥₑ−ratio_{negative}). Inhibition (%)=100−Activation (%). The positive control was the whole reaction system containing PARP7 enzyme, RBN011147, MAb Anti His-Tb cryptate Gold, and Streptavidin-d2, but with DMSO instead of compound. The negative control was the whole reaction system containing RBN011147, MAb Anti His-Tb cryptate Gold, Streptavidin-d2, and DMSO instead of compound, with no PARP7 enzyme.

The IC₅₀ value was obtained by 4 Parameter Logistic (4PL 1/y2) model fitting, and the measured results are shown in Table 3:

TABLE 3

| Compound | PARP7 Enzyme (IC₅₀ μM) |
|---|---|
| Compound I | 0.001582 |

From Table 3, it can be seen that compound I of the present invention have good inhibitory effect on PARP7 enzyme.

### 2. Lung Cancer Cell Proliferation Inhibition Experiment

In this experiment, the CTG method was used to test the inhibition of the compounds on the proliferation of lung cancer cell line H1373 (high expression of PARP7), and half inhibitory concentration IC₅₀ of the compound to H1373 was obtained. The H1373 cell line was purchased from ATCC, the complete culture medium was ATCC modified RPMI 1640 medium+10% FBS (Fetal bovine serum)+1% PS (Penicillin-Streptomycin Liquid). RPMI 1640 cell culture medium, fetal bovine serum, and trypsin were purchased from Gibco, and cell culture flasks were purchased from Greiner, disposable Cell Counting Plate, and trypan Blue Solution purchased from Bio-Rad.

(1) 100 μl of H1373 cells suspension was seeded in a 96-well cell culture plate, and the density of the suspension in each well was 1.5×10⁴ cells/ml. The culture plate was incubated in incubator for 16-24 h (37° C., 5% CO₂);

(2) The solution of each compound to be tested with different concentration was obtained using gradient dilution. 2 μl of the solution of each compound to be tested was mixed with 198 μl RPMI 1640 containing 1% PS to obtain the final solution. The final solution was transferred to the culture plate (25 μl/well, 2 parallel wells per concentration), and the culture plate was incubated for 144 hrs in incubator (37° C., 5% CO₂). Cell Titer Glo reagent was added into each well of the culture plate, and then the culture plate was shaken for 2 mins and incubated for an additional 10 mins at room temperature.

(3) The luminescence signal of each well was measured on SPARK plate reader.

(4) The inhibition rate was calculated by the luminescence signal value.

(5) The curve was fitted with the inhibition rate of different concentrations, and then the IC₅₀ of compounds were calculated.

The measured results were shown in Table 4:

TABLE 4

| Compound | H1373 (IC₅₀ μM) |
|---|---|
| Compound I | 0.013012 |

From the Table 4, it can be seen that the representative compounds of the present invention have good inhibitory effect on the proliferation of H1373 cell.

### Example 2. Preparation and Characteristics of the Crystalline Form 1 of Compound I

Compound I (30.98 g, 0.057 mol) as white foam which is synthesized in Example 1 was added to ethanol (300 mL) and stirred until the solid is dissolved clearly at room temperature. The resulting solution was added water (300 mL) dropwise. The mixture is stirred for 1.5 h and filtered. The filter cake is dried under vacuum at 40° C. for 20 h to give the crystalline Form 1 of Compound I (26.67 g, 86% yield) as a white solid. And the crystalline Form I of Compound I was analyzed by XRPD pattern, DSC, TGA, and DVS with the following general method.

### General Method 1: X-Ray Powder Diffraction (XRPD)

The XRPD pattern data in the present invention were collected according to the following general protocol.

### Instrument, Parameters and Method:

The XRPD was conducted for each sample using a Bruker D8 Advance Diffractometer. The X-ray tube voltage and amperage were set to 40 kV and 40 mA respectively. Data was collected using Collection Software (Diffrac Plus XRD Commander) at the Cu Kα radiation with a wavelength of 1.54 Å from 3.0 to 40 degrees (2θ)/3.0 to 30 degrees (2θ) using a step size of 0.02° (2θ) degrees and a step time of 0.2 seconds. A typical error associated with measurement can occur as a result of a variety of factors. Therefore peaks are considered to have a typical associated error of ±0.2≥ 2θ.

The model of the zero background sample holder is 24.6 mm diameter×1.0 mm thickness, manufactured by MTI Corporation. Unless indicated otherwise, the sample hasn't been ground before testing.

### Peak Selection Method:

The XRPD patterns collected were imported into MDI Jade. The measured XRPD pattern was aligned to a pattern of a sample with an internal reference to determine the absolute peak positions of the sample. The internal reference used was corundum and the absolute peak position for corundum were calculated based on the corundum cell parameters. All peaks of the sample were extracted in a table with the accurate peak position together with the relative peak intensities. A typical error of ±0.2° 2θ in peak positions applies to this data. The minor error associated with this measurement can occur as a result of a variety of factors including:
(a) sample preparation (e.g., sample height);
(b) instrument;
(c) calibration;
(d) operator (including those errors present when determining the peak locations), and
(e) the nature of the material (e.g. preferred orientation and transparency errors).

Therefore peaks are considered to have a typical associated error of ±0.2° 2θ. When a higher intensive peak has a shoulder peak, no matter the 2θ difference value between the higher intensive peak and the shoulder peak are less or more than 0.2° 2θ, the higher intensive peak has been preferably selected as the characteristic peak, and the lower intensive shoulder peak has not been selected as the characteristic peak.

### General Method 2: Differential Scanning Calorimetry (DSC)

DSC analyses were carried out with a TA Instruments Q200 DSC. The sample pan is aluminium covered without a hole. The sample weight is 0.5 mg˜5 mg. The sample were analyzed from the equilibrated temperature 0° C. to the highest test temperature 300° C. or 350° C. at a heating rate of 10° C./min under 50 mL/min nitrogen flow.

### General Method 3: The Thermogravimetric Analysis (TGA)

TGA measurements were performed using a TA Instruments Q500 TGA using nitrogen purge gas at a rate of 40 ml/min (Hi-Res sensitivity 3.0; Ramp 10.00° C./min, res 5.0 to 150.00° C.; Ramp 10.00° C./min to 350° C.). The sample pan is a platinum pan. The sample weight is 1 mg˜10 mg.

### General Method 4: The Dynamic Vapor Sorption Analysis (DVS)

DVS analyses were carried out with an Intrinsic PLUS. The sample pan is stainless pan. The sample weight is 58.24 mg. The sample were analyzed from the equilibrated temperature 25° C., at humidity 0%; isothermal for 90 min; abort next iso if weight (%)<0.0100 for 15.00 min; step humidity 10% every 90 min to 80%; abort next iso if weight (%)<0.0100 for 15.00 min; step humidity 10% every 90 min to 0% at a rate of 200 sccm nitrogen flow.

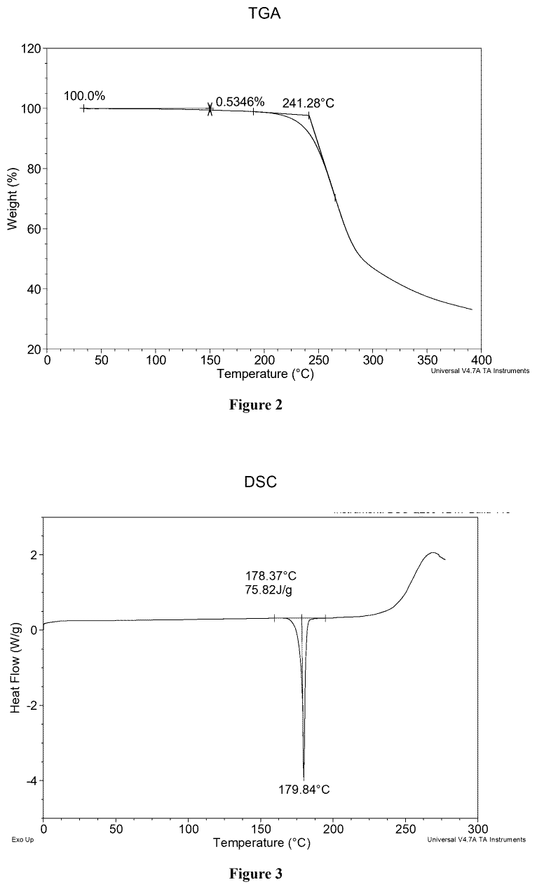

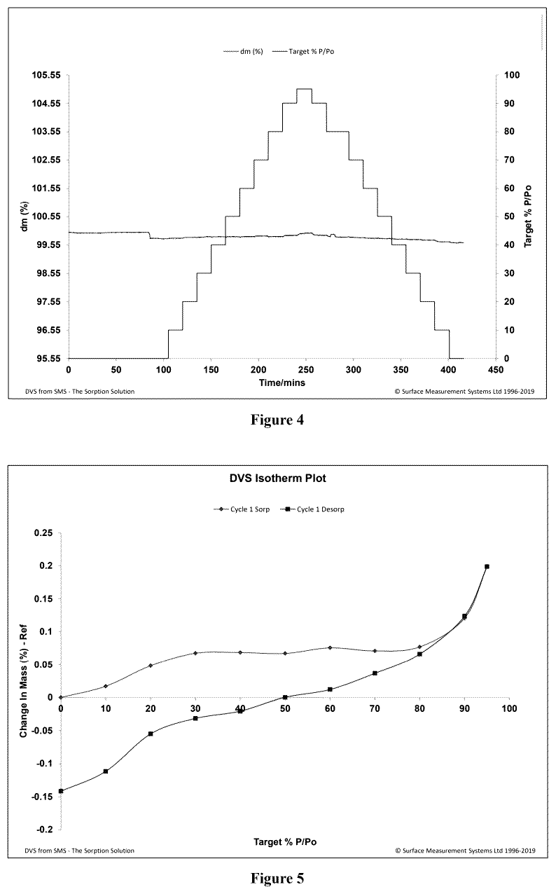

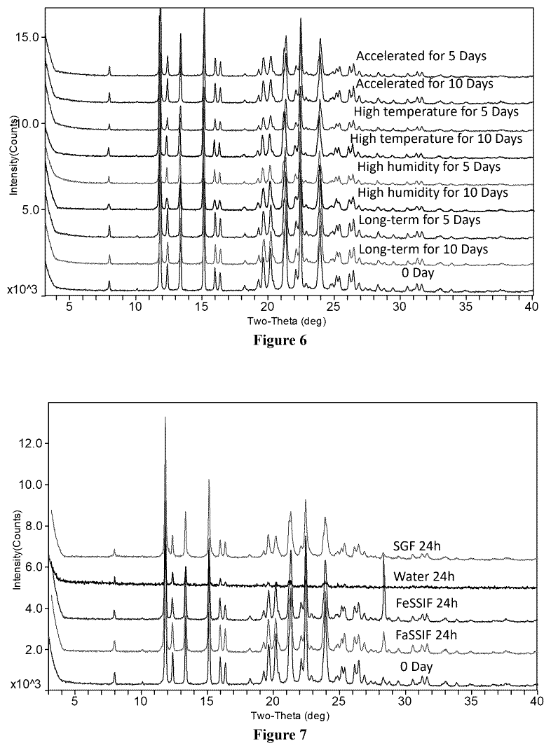

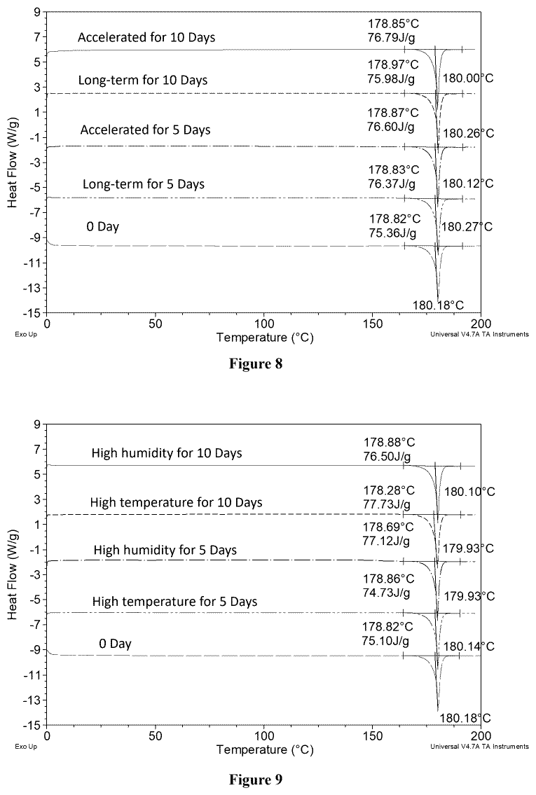

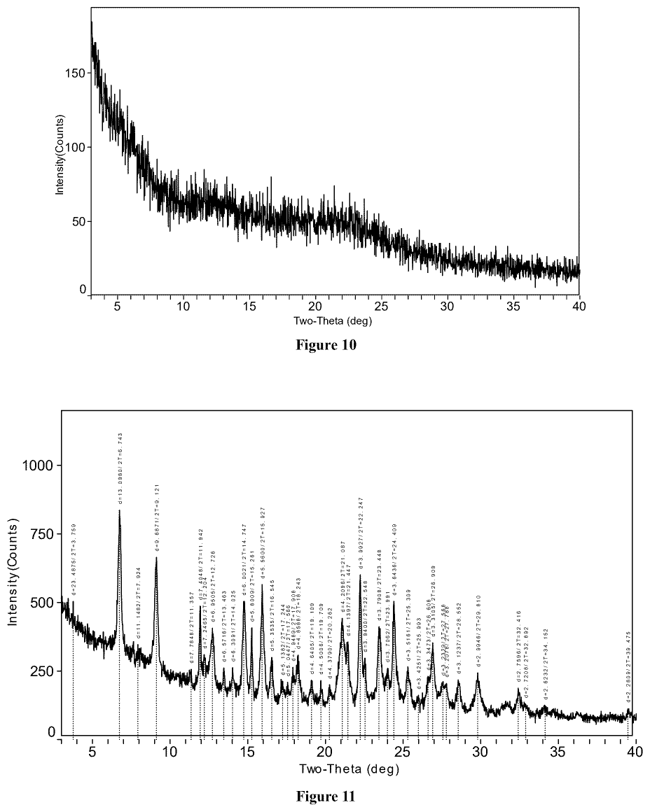

The results are displayed in FIG. 1-FIG. 5 respectively. Some characteristic peaks in the XRPD pattern are listed in Table 5.

TABLE 5

| 2 theta | d spacing | Intensity % |
|---|---|---|
| 8.12 | 10.88 | 5.4 |
| 11.96 | 7.40 | 100 |
| 12.49 | 7.08 | 13.8 |
| 13.48 | 6.56 | 30.7 |
| 15.27 | 5.80 | 58.7 |
| 16.11 | 5.50 | 12.5 |
| 16.49 | 5.37 | 12.1 |
| 19.79 | 4.48 | 25.4 |
| 20.30 | 4.37 | 35 |
| 21.45 | 4.14 | 65.2 |
| 22.59 | 3.93 | 83 |
| 24.06 | 3.70 | 58.4 |

### Example 3. Stability Evaluation of Crystalline Form 1 of Compound I

The stability of crystalline Form 1 of Compound I was tested according to the following table 6.

TABLE 6
Solid State Stability Information of crystalline form 1 of Compound I

| Items | Content | |
|---|---|---|
| Experimental | Long-term (CQ) | (25° C. ± 5° C., 60% RH ± 5%) Exposed, dark |
| Conditions | Accelerated (JS) | (40° C. ± 2° C., 75% RH ± 5% RH) Exposed, dark |
| | High temperature (GW) | (50° C. ± 5° C., <30% RH) Exposed, dark |
| | High humidity (GS) | (25° C. ± 2° C., 90% RH ± 5% RH) Exposed, dark |

| | |
|---|---|
| Test Time | 0 day, 5 days and 10 days |
| Test Items | Polymorph (via XRPD), Melting point (via DSC), Related substances |
| | (via HPLC) |
| Results | The polymorph remained unchanged under above 4 conditions for 10 |
| | days. |
| | Under above 4 conditions for 10 days, the melting point range of |
| | Compound I was 179° C. ± 1° C. |
| | Under above 4 conditions for 10 days, the purity decrease of |
| | Compound I was less than 0.1%. |

The solid stability of crystalline form 1 of Compound I was exposure for 10 days in high humidity (25° C.±2° C., 90% RH±5% RH), high temperature (50° C.±5° C., <30% RH), long-term (25° C.±5° C., 60% RH±5%) and accelerated (40° C.±2° C., 75% RH±5%) conditions. The results suggest that Form 1 remain unchanged, that the melting point is 179±1° C., and that the purity decrease less than 0.1%.

### Example 4. Equilibrium Solubility Crystalline Form 1 of Compound I

The equilibrium solubility of crystalline form 1 of Compound I was tested according to the following table 7.

TABLE 7
Experimental Information of Equilibrium Solubility
on crystalline form 1 of Compound I

| Items | Content |
|---|---|
| Polymorph | Form 1 |
| Medium | Water, SGF, FeSSIF, FaSSIF |
| Temperature | 25° C., 37° C. |
| Test Time | 4 h, 24 h |
| Test Items | Solubility (via HPLC); Polymorph (via XRPD, if the solid was sufficient |
| | for test after 24 h) |

The equilibrium solubility test results of the crystalline form 1 of Compound I suggest:

(1) The solubility of the crystalline form 1 of Compound I in FeSSIF is slightly higher, about 0.1 mg/mL.

(2) The crystalline form 1 of Compound I is to reach basic saturation after 4 h in water (25° C.), SGF (37° C.), FaSSIF (37° C.), FeSSIF (37° C.).

(3) The crystalline Form 1 of form 1 of Compound I remains unchanged after 24 h in water (25° C.), SGF (37° C.), FaSSIF (37° C.), FeSSIF (37° C.).

### Example 5. Preparation and Characteristics of an Amorphous Compound I

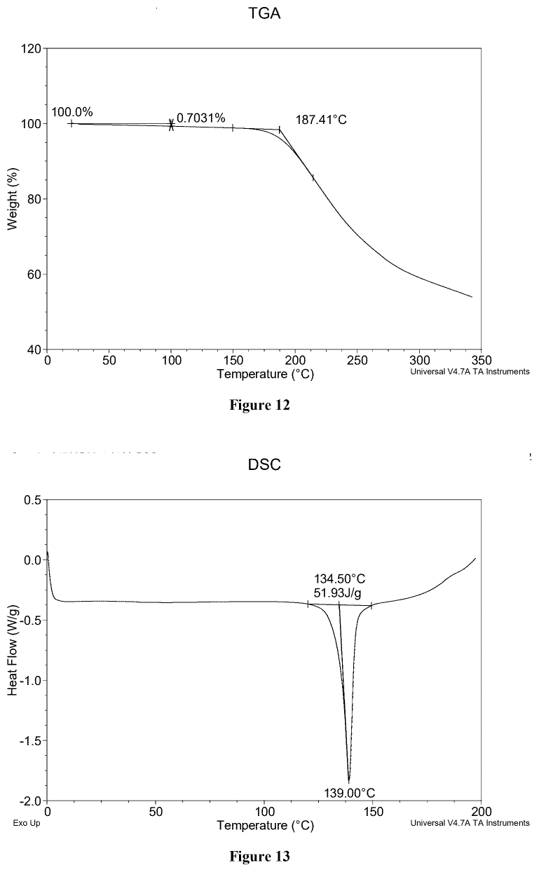

About 1.5 g Compound I were dissolved in 10 mL acetone by applying ultrasound, and concentrated under reduced pressure at 50° C. to obtain the amorphous form Compound I. The amorphous Compound I was analyzed by XRPD pattern, with the above general method 1. The results are displayed in FIG. 12.

### Example 6. Preparation and Characteristics of the Crystalline Form a of Tosilate of Compound I

Step 1: About 200 mg of Compound I as white foam was dissolved in 5.0 mL ethyl acetate to obtain solution 1.

Step 2: About 69 mg tosic acid (p-toluenesulfonic acid), was dissolved in 0.2 mL Ethanol to obtain solution 2.

Step 3: Under stirring at room temperature, Solution 2 was added to Solution 1, to obtain solution 3.

Step 4: Under stirring at room temperature for 0.5 h, precipitation not occurred, then stirred overnight at 4° C., precipitation also not occurred, 15.0 mL n-Heptane was added, precipitation occurred, then stirred overnight at 4° C., precipitation not disappeared.

Step 5: The precipitation was centrifuged and dried under vacuum at room temperature overnight to obtain tosilate Form A.

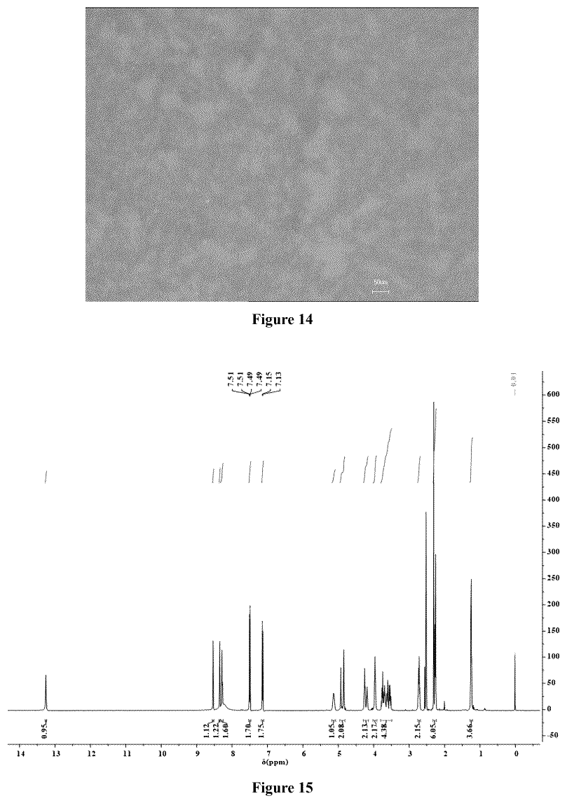

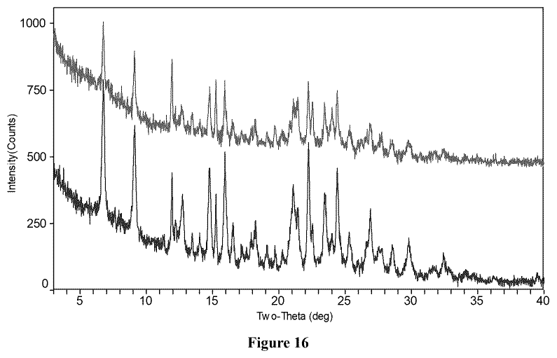

Step 6: Analyzed by XRPD pattern, DSC, TGA, PLM and 1H-NMR with the general method, and the result was shown in FIG. 11-16.

### Example 7. Hygroscopicity Evaluation of the Crystalline Form A of Tosilate of Compound I

The hygroscopicity of the crystalline Form A of tosilate of Compound I was tested and the result was shown in FIG. 16.

Each of the references including all patents, patent applications and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

1. A form of Compound I, which is selected from the group consisting of:
A crystalline of Compound I;
A salt of Compound I with an acid; and
A crystalline of the salt of Compound I with an acid;
Wherein, the Compound I is 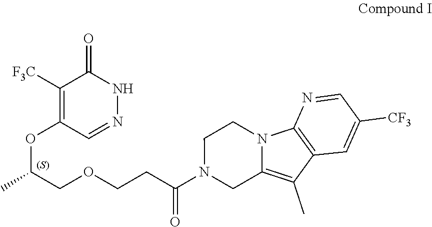
The acid is selected from the group consisting of hydrochloric acid, sulfuric acid, hydrobromic acid, methanesulfonic acid, tosic acid, oxalic acid, maleic acid, phosphoric acid, L-tartric acid, fumaric acid, citric acid, lactobionic acid, mandelic acid, L-malic acid, hippuric acid, L-lactic acid, succinic acid, benzoic acid, adipic acid, and acetic acid.

2. The form of Compound I according to claim 1, wherein, the crystalline of Compound I is a crystalline Form 1 of Compound I, and the crystalline Form 1 of Compound I is characterized by X-ray powder diffraction pattern comprising characteristic peaks at 2θ values of 8.12±0.2°, 11.96±0.2°, 13.48±0.2° and 15.27±0.2°.

3. The form of Compound I according to claim 2, wherein the X-ray powder diffraction pattern further comprises one or more characteristic peaks at 2θ values selected from the group consisting of 16.11±0.2°, 16.49±0.2°, 19.79±0.2°, and 20.30±0.2°.

4. The form of Compound I according to claim 2, wherein the X-ray powder diffraction pattern further comprises:
one or two characteristic peaks at 2θ value selected from the group consisting of 19.79±0.2° and 20.30±0.2°, and
one or two characteristic peaks at 2θ values selected from the group consisting of 16.11±0.2° and 16.49±0.2°.

5. The form of Compound I according to claim 2, wherein the X-ray powder diffraction pattern further comprises characteristic peaks at 2θ value selected from the group consisting of:
16.11±0.2° and 16.49±0.2°;
16.11±0.2°, 16.49±0.2° and 20.30±0.2°;
16.11±0.2°, 16.49±0.2° and 19.79±0.2°;
19.79±0.2° and 20.30±0.2°;
16.11±0.2°, 19.79±0.2° and 20.30±0.2°; and
16.49±0.2°, 19.79±0.2° and 20.30±0.2°.

6. The form of Compound I according to claim 2, wherein the X-ray powder diffraction pattern comprises characteristic peaks at 2θ values of 8.12±0.2°, 11.96±0.2°, 13.48±0.2°, 15.27±0.2°, 16.11±0.2°, 16.49±0.2°, 19.79±0.2° and 20.30±0.2°.

7. The form of Compound I according to claim 2, wherein the X-ray powder diffraction pattern further comprises one or two characteristic peaks at 2θ values selected from the group consisting of 12.49±0.2° and 21.45±0.2°.

8. The form of Compound I according to claim 2, wherein the X-ray powder diffraction pattern comprises characteristic peaks at 2θ values of 8.12±0.2°, 11.96±0.2°, 12.49±0.2°, 13.48±0.2°, 15.27±0.2°, 16.11±0.2°, 16.49±0.2°, 19.79±0.2°, 20.30±0.2° and 21.45±0.2°.

9. The form of Compound I according to claim 2, wherein the X-ray powder diffraction pattern further comprises one or two characteristic peaks at 2θ values selected from the group consisting of 22.59±0.2° and 24.06±0.2°.

10. The form of Compound I according to claim 2, wherein the X-ray powder diffraction pattern comprises the characteristic peaks at 2θ values of 8.12±0.2°, 11.96±0.2°, 12.49±0.2°, 13.48±0.2°, 15.27±0.2°, 16.11±0.2°, 16.49±0.2°, 19.79±0.2°, 20.30±0.2°, 21.45±0.2°, 22.59±0.2° and 24.06±0.2°.

11. The form of Compound I according to claim 2, wherein the X-ray powder diffraction pattern is the same as FIG. 1.

12. The form of Compound I according to claim 1, wherein the acid is tosic acid.

13. The form of Compound I according to claim 12, wherein the crystalline of the salt of Compound I with an acid is the crystalline Form A of the salt of Compound I with tosic acid and is characterized by X-ray powder diffraction pattern comprising characteristic peaks at 2θ values of 6.74±0.2°, 9.12±0.2°, 14.75±0.2° and 15.93±0.2°.

14-21. (canceled)

22. A pharmaceutical composition comprising a therapeutically effective amount of the form of Compound I according to claim 1, and at least one pharmaceutically acceptable excipient.

23-25. (canceled)

26. A method of treating a subject having a cancer related to PARP7, said method comprising administering to the subject a therapeutically effective amount of the form of Compound I according to claim 1.

27. The method according to claim 26, wherein, the cancer related to PARP7 is PARP7 overexpression associated cancer.

28. The method according to claim 26, wherein, the cancer is selected from the group consisting of breast cancer, cancer of the central nervous system, endometrium cancer, kidney cancer, large intestine cancer, lung cancer, esophagus cancer, tongue cancer, ovarian cancer, pancreatic cancer, prostate cancer, stomach cancer, mesothelioma, melanoma, fibrosarcoma, bladder cancer, rectal cancer, lymphoma, cervical cancer, head and neck cancer, upper aerodigestive cancer, colorectal cancer, urinary tract cancer, and colon cancer.

29-31. (canceled)

32. The form of Compound I according to claim 2, wherein, the crystalline Form 1 of Compound I is characterized by X-ray powder diffraction pattern comprising characteristic peaks at 2θ values of Table 1:
TABLE 1

| 2 theta | d spacing | Intensity % |
|---|---|---|
| 8.12 | 10.88 | 5.4 |
| 11.96 | 7.40 | 100 |
| 12.49 | 7.08 | 13.8 |
| 13.48 | 6.56 | 30.7 |
| 15.27 | 5.80 | 58.7 |
| 16.11 | 5.50 | 12.5 |
| 16.49 | 5.37 | 12.1 |
| 19.79 | 4.48 | 25.4 |
| 20.30 | 4.37 | 35 |
| 21.45 | 4.14 | 65.2 |
| 22.59 | 3.93 | 83 |
| 24.06 | 3.70 | 58.4 |

33. The method according to claim 28, each cancer is independently selected from the group consisting of adenocarcinoma, squamous cell carcinoma, mixed adenosquamous carcinoma, and undifferentiated carcinoma.

34. The method according to claim 28, wherein:
the ovarian cancer is selected from the group consisting of high grade ovarian serious adenocarcinoma, ovarian mucinous cystadenocarcinoma, and malignant ovarian Brenner tumor;
the kidney cancer is clear cell renal cell carcinoma;
the tongue cancer is tongue squamous cell carcinoma;
the lung cancer is selected from the group consisting of lung adenocarcinoma, lung adenosquamous carcinoma, squamous cell lung carcinoma, large cell lung carcinoma, small cell lung carcinoma, papillary adenocarcinoma of the lung, and non-small cell lung carcinoma;
the pancreatic cancer is selected from the group consisting of pancreatic adenocarcinoma, and pancreatic ductal adenocarcinoma;
the esophagus cancer is esophageal squamous cell carcinoma;
the mesothelioma is biphasic mesothelioma;
the cancer of the central nervous system is selected from the group consisting of neuroglioma, glioblastoma, and glioblastoma multiforme;
the stomach cancer is gastric adenocarcinoma;
the breast cancer is selected from the group consisting of ductal breast carcinoma, breast adenocarcinoma, and HR+ breast cancer;
the bladder cancer is bladder squamous cell carcinoma;
the melanoma is malignant melanoma;
the colon cancer is colon adenocarcinoma;
the head and neck cancer is head and neck small squamous cell cancer.
